(19) United States
(12) Patent Application Publication
(10) Pub. No.: US 2023/0166236 A1
(43) Pub. Date: Jun. 1, 2023

(54) Enrichment and Purification of Specific Compounds from Hydrocarbon Reservoir Produced Water using Mixed-Mode Solid Phase Extraction

(71) Applicant: Saudi Arabian Oil Company, Dhahran (SA)

(72) Inventors: Wei Wang, Quincy, MA (US); Sehoon Chang, Boston, MA (US); Hooisweng Ow, Woburn, MA (US); Gawain Thomas, Shirley, MA (US)

(21) Appl. No.: 17/539,900

(22) Filed: Dec. 1, 2021

(51) Int. Cl. B01J 20/26; B01D 15/10; B01J 20/28; G01N 1/40

(52) U.S. Cl. CPC B01J 20/267; B01D 15/10; B01J 20/28014; G01N 1/405; G01N 33/24

(57) ABSTRACT

Compositions and methods for extracting tracers from subterranean fluids are provided. The compositions include a media for solid phase extraction. The media includes a homogenous mixture of a first and second sorbent. The first sorbent includes a crosslinked polystyrene divinyl benzene copolymer core functionalized with hydroxylated polystyrene divinyl benzene copolymer. The second sorbent includes a crosslinked polystyrene divinyl benzene core functionalized with a copolymer that includes secondary and tertiary amino groups. A solid phase extraction cartridge including a homogenous mixture of the first and second sorbent is also provided. Methods of extracting tracers from subterranean fluids include extracting the tracers with a solid phase extraction column that includes a homogenous mixture of the first and second sorbent.

### TECHNICAL FIELD

This document relates to solid phase extraction of compounds of interest from wellbore produced fluids or subterranean fluids.

### BACKGROUND

Solid phase extraction (SPE) is a method of purifying molecular compounds from a fluid using a solid phase sorbent. For example, a fluid is passed over the solid phase sorbent. Compounds of interest or undesired impurities bind to the sorbent and the remaining fluid flows through the solid phase. Further washing steps can elute the compound of interest or impurities from the solid phase.

### SUMMARY

This disclosure describes compositions and methods for recovering tracers from oil field produced waters and other fluids associated with wellbores and subterranean formations.

In some implementations, a media for solid phase extraction includes a homogenous mixture of a first sorbent and a second sorbent. The first sorbent comprises a first polymer core. The first polymer core includes a crosslinked polystyrene divinyl benzene copolymer core functionalized with a hydroxylated polystyrene divinyl benzene copolymer. The second sorbent includes a second polymer core. The second polymer core includes a crosslinked polystyrene divinyl benzene copolymer core functionalized with a copolymer that contains secondary and tertiary amino groups.

This aspect, taken alone or combinable with any other aspect, can include the following features. The homogenous mixture of the first sorbent and the second sorbent includes an equal amount of the first sorbent and the second sorbent.

This aspect, taken alone or combinable with any other aspect, can include the following features. The homogenous mixture of the first sorbent and the second sorbent includes a 2:5 ratio by weight of the first sorbent to the second sorbent.

In some implementations, a solid phase extraction cartridge includes an extraction column, a filter at a bottom of the extraction column, and a solid phase media in the extraction column. The solid phase media includes a homogenous mixture of a first sorbent and a second sorbent. The first sorbent includes a first polymer core. The first polymer core includes a crosslinked polystyrene divinyl benzene copolymer core functionalized with a hydroxylated polystyrene divinyl benzene copolymer. The second sorbent includes a second polymer core. The second polymer core includes a crosslinked polystyrene divinyl benzene copolymer core functionalized with a copolymer that contains secondary and tertiary amino groups.

This aspect, taken alone or combinable with any other aspect, can include the following features. The homogenous mixture of the first sorbent and the second sorbent includes an equal amount of the first sorbent and the second sorbent.

This aspect, taken alone or combinable with any other aspect, can include the following features. The homogenous mixture of the first sorbent and the second sorbent includes a 2:5 ratio by weight of the first sorbent to the second sorbent.

In some implementations, a method of extracting tracer compounds from a fluid sample includes providing a solid phase extraction cartridge. The solid phase extraction cartridge includes an extraction column, a filter at a bottom of the extraction column, and a solid phase media in the extraction column. The solid phase media includes a homogenous mixture of a first sorbent and a second sorbent. The first sorbent includes a first polymer core. The first polymer core includes a crosslinked polystyrene divinyl benzene copolymer core functionalized with a hydroxylated polystyrene divinyl benzene copolymer. The second sorbent includes a second polymer core. The second polymer core includes a polystyrene divinyl benzene copolymer core functionalized with a copolymer that contains secondary and tertiary amino groups. The method includes flowing the fluid sample through the solid phase extraction cartridge, and extracting tracer compounds from the solid phase extraction cartridge using an eluent.

This aspect, taken alone or combinable with any other aspect, can include the following features. Extracting the tracer compounds from the solid phase extraction cartridge using an eluent includes extracting the tracer compounds from the solid phase extraction cartridge using methanol.

This aspect, taken alone or combinable with any other aspect, can include the following features. The method includes extracting the tracer compounds from the solid phase extraction cartridge using a second eluent.

This aspect, taken alone or combinable with any other aspect, can include the following features. Extracting the tracer compounds from the solid phase extraction cartridge using a second eluent includes extracting the tracer compounds from the solid phase extraction cartridge using a solution of ammonia in methanol.

This aspect, taken alone or combinable with any other aspect, can include the following features. The method includes conditioning the solid phase extraction cartridge with methanol before flowing the fluid sample through the solid phase extraction cartridge.

This aspect, taken alone or combinable with any other aspect, can include the following features. The method includes conditioning the solid phase extraction cartridge with deionized water before flowing a fluid sample through the solid phase extraction cartridge.

This aspect, taken alone or combinable with any other aspect, can include the following features. The method includes washing the solid phase extraction cartridge between flowing the fluid sample through the solid phase extraction cartridge and extracting the tracer compounds from the solid phase extraction cartridge using an eluent. Washing the solid phase extraction cartridge includes flowing deionized water through the cartridge.

This aspect, taken alone or combinable with any other aspect, can include the following features. The method includes analyzing the extracted tracer compounds using HPLC, UV/Vis spectroscopy, or both.

In some implementations, a method of recovery tracers from a subterranean fluid includes recovering a fluid sample from a subterranean formation. The fluid sample includes tracer compounds. The method includes providing a solid phase extraction cartridge. The solid phase extraction cartridge includes an extraction column, a filter at a bottom of the extraction column, and a solid phase media in the extraction column. The solid phase media includes a homogenous mixture of a first sorbent and a second sorbent. The first sorbent comprises a first polymer core. The first polymer core includes a crosslinked polystyrene divinyl benzene copolymer core functionalized with a hydroxylated polystyrene divinyl benzene copolymer. The second sorbent includes a second polymer core. The second polymer core includes a crosslinked polystyrene divinyl benzene copolymer core functionalized with a copolymer that contains secondary and tertiary amino groups. The method includes flowing the fluid sample through the solid phase extraction cartridge, and extracting the tracer compounds from the solid phase extraction cartridge using an eluent.

The details of one or more implementations of the disclosure are set forth in the accompanying drawings and the description that follows. Other features, objects, and advantages of the disclosure will be apparent from the description and drawings, and from the claims.

### DESCRIPTION OF DRAWINGS

FIG. 1 shows an example schematic of an SPE cartridge.

FIG. 2 shows an example flowchart of a method of extracting tracer compounds from a fluid sample.

FIG. 3 shows an example flowchart of a method of recovering tracers from a subterranean fluid.

FIG. 4 shows an example HPLC UV/Vis analysis of the water sample before and after sequential SPE extraction.

FIG. 5 shows an example schematic of a layered SPE cartridge.

FIG. 6 shows an example analysis of the recovery of the tracers from a layered SPE cartridge.

FIG. 7 shows an example schematic of an SPE extraction using a homogenously mixed solid phase bed.

FIG. 8 shows an example HPLC UV/Vis analysis of the combined eluents of a SPE extraction using a homogenously mixed solid phase bed.

FIG. 9 shows an example of an HPLC UV/Vis analysis of the recovery of tracer materials passed over an Oasis WAX cartridge.

FIG. 10A shows an example HPLC UV/Vis analysis of recovered tracers in oil field produced water, recovered by a homogenously mixed ENV+/HR-XAW SPE cartridge, compared to a reference sample of tracers in produced water.

FIG. 10B shows an example HPLC UV/Vis analysis of recovered tracers in oil field produced water, recovered by an Oasis WAX SPE cartridge, compared to a reference sample of tracers in produced water.

FIG. 11A shows an example HPLC UV/Vis analysis of seawater with tracers over a homogenously mixed ENV+/HR-XAW cartridge and an Oasis WAX cartridge.

FIG. 11B shows an example HPLC UV/Vis analysis of tracers recovered from a low salinity brine with a homogenously mixed ENV+/HR-XAW cartridge and an Oasis WAX cartridge.

Like reference symbols in the various drawings indicate like elements.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Provided in this disclosure, in part, are methods, compositions, and systems for extracting tracers from oil field produced water, fluid produced from a hydrocarbon reservoir, or other fluids associated with wellbores and subterranean formations. In more detail, provided herein are solid phase sorbents and methods of solid phase extraction (SPE) to purify molecular compounds of interest from a fluid. The fluid can be, for example, a produced oil filed water or other wellbore or subterranean fluid. The fluid can also include dissolved organic matters and salts. The SPE sorbents and methods described herein can be used to preconcentrate and extract tracers from these fluids.

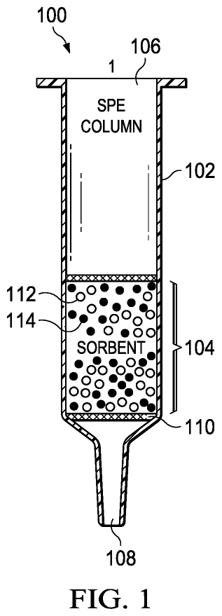

Solid phase extraction can take place in a cartridge. FIG. 1 shows an example schematic of an SPE cartridge 100 that includes a column 102 and a solid sorbent media bed 104. The solid sorbent media bed 104 includes a homogenous mixture of a first sorbent 112 and a second sorbent 114. The first sorbent 112 includes functionalized, spherical copolymer beads. The beads include a crosslinked polystyrene divinyl benzene copolymer core functionalized with hydroxylated polystyrene divinyl benzene copolymer. The second sorbent 114 also includes functionalized, spherical copolymer beads. The beads include a crosslinked polystyrene divinyl benzene core, functionalized with a copolymer that includes secondary and tertiary amino groups. The cartridge has an upper opening 106 and a lower spout 108. In some implementations, the cartridge includes a filter 110 that holds the solid sorbent in place when the cartridge is upright. For example, the filter can be a polyethylene (PE) frit (filter) with 10 μm porosity.

A process for recovering tracers from a sample can include the following steps. First, a liquid sample including the tracers is passed over, i.e., poured into the upper opening of a cartridge containing solid sorbent media. The fluid flows through the cartridge and the tracers bind to the solid sorbent media. In some implementations, the initial flow-through, i.e., the liquid that passes through the column, is discarded. In some implementations, the cartridge is then washed, for example, with water, methanol, or another solvent. The washing steps do not displace the tracers from the solid sorbent media, but can rinse unbound materials, salts, or remaining fluid sample from the cartridge. Next, the tracer compounds are displaced from the solid sorbent media using a solvent or eluent. The organic solvent or eluent can be, for example, methanol, 25 nM ammonia in methanol, or methanolic acid (0.5 M HCl in methanol). The resulting eluate includes the tracers in a concentrated form that can be subsequently analyzed, for example by HPLC, UV/Vis spectroscopy, or mass spectrometry. The SPE process therefore is beneficial for detecting tracers present in a sample, even in small amounts. Advantageously, the SPE process enriches the amount of molecular compounds, typically by 100× or more. In some instances, this raises the concentration of the compounds from below the detection limits to within the detection limits of subsequent analytical techniques, for example HPLC UV/Vis analysis or mass spectroscopy.

Further, the SPE processes described herein increase throughput and decrease cycle time by accomplishing purification and enrichment steps in a single extraction. This is advantageous and more efficient compared to a recovery procedure that requires multiple extractions or multiple SPE cartridges.

The SPE processes described herein are used to extract and concentrate water tracer molecules. These water tracers can be used to trace fluid flow in a subterranean formation. For example, inter-well tracers can be injected into a first subterranean location, recovered at second subterranean location, and used to elucidate well connectivity and to optimize production via active rate adjustments in waterflooding campaigns. The tracers include derivatives of dipicolinic acid, derivatives of phenanthroline dicarboxylic acids, derivatives of sulfonated naphthalenes and pyrenes, water soluble sulfonated and/or carboxylated derivatives of xanthenes. Herein, the compounds dipicolinic acid (DPA), chelidamic acid (CDA), 4-chloropyridine dicarboxylic acid (Cl-DPA), and 1,5-naphthalene disulfonate disodium (1,5-NDS) were used as representative water tracers to demonstrate the efficiency of the SPE processes. These water tracers represent chemically distinct families that have different chemical functional groups and affinities. Purifying a mixture that contains a mixture of unique tracers can be difficult. In some implementations, a method could include passing a sample over multiple types of sorbent media sequentially. For example, a method could include passing a sample over a first cartridge functionalized to capture a first tracer and eluting the first tracer from the cartridge, passing the sample over a second cartridge functionalized to capture a second tracer and eluting the second tracer from the cartridge, and reiterating for as many tracers as desired. However, this approach is time consuming and can result in material loss, i.e., a low recovery of the tracer compounds. Further, automation of a process that includes multiple separate SPE cartridges is difficult. A single SPE cartridge that can purify and preconcentrate a large variety of tracers is therefore desirable.

Provided in this disclosure is a homogenously mixed mode SPE cartridge 100, which contains a homogenous mixture of a first sorbent 112 and a second sorbent 114, wherein each sorbent has a different chemical functionality. Advantageously, this cartridge provides high throughput, minimizes materials losses, and has a high recovery factor. Further, as only a single cartridge is required, the process can be easily automated.

In a homogenously mixed-mode SPE cartridge 100, two different sorbents with different chemical functionalities are homogenously mixed. As described herein, the first sorbent 112 includes functionalized, spherical copolymer beads. The beads include crosslinked polystyrene divinyl benzene copolymer functionalized with a hydroxylated polystyrene divinyl benzene copolymer. The first sorbent is a water wettable sorbent, due to the hydroxylated functionalization and the retention that occurs due to hydrophobic interactions between the sorbent and tracer molecules. An example of this type of sorbent is the reversed phase mode non-polar sorbent sold under the trademark INSOLUTE® ENV+. Herein, INSOLUTE® ENV+(ENV+) is used as a representative of a sorbent that includes a crosslinked polystyrene divinyl benzene copolymer core functionalized with hydroxylated polystyrene divinyl benzene copolymer.

The second sorbent 114 also includes functionalized, spherical copolymer beads. The beads include a crosslinked polystyrene divinyl benzene copolymer core, functionalized with secondary and tertiary amino groups. An example of this type of sorbent is the secondary and tertiary ammonium modified crosslinked polystyrene divinyl benzene copolymer sold under the tradename Chromabond® HR-XAW sorbents. Herein, Chromabond® HR-XAW (HR-XAW) sorbents are used as a representative of crosslinked polystyrene divinyl benzene sorbents functionalized with secondary and tertiary amine groups.

Provided herein is an SPE cartridge 100 that contains a homogenous mixture of both types of sorbents. In some implementations, the weight ratio of first sorbent to second sorbent is between 1:10 first sorbent to second sorbent and 10:1 first sorbent to second sorbent. In some implementations, the ratio of first sorbent to second sorbent is 1:2.5 by weight. In some implementations, the first and second sorbents are present in equal amounts. In some implementations, the ratio of first sorbent to second sorbent can vary based on the types of tracer materials to be extracted by weak anion exchange versus hydrophobic interactions. In more detail, the cartridge contains a homogenously mixed sorbent media that includes a first sorbent 112 with hydroxyl functional groups on a crosslinked polystyrene divinyl benzene copolymer core and a second sorbent 114 that includes secondary and tertiary amino groups. The homogenously mixed sorbent media efficiently extracts and concentrates several tracer compounds of interest. The homogenously mixed sorbent media efficiently extracts 1,5-NDS, CDA, DPA, and Cl-DPA. As described herein, the homogenously mixed sorbent media outperforms several other configurations in terms of tracer recovery. For example, the homogenously mixed sorbent media recovers more of each type of tracer than either type of sorbent media alone. Further, the homogenously mixed sorbent media has a higher recovery of tracer compounds, compared to sequential SPE extraction using a first cartridge with the first media and a subsequent extraction using a second cartridge with the second media, or compared to three sequential SPE extractions using a first cartridge with the first media, a second cartridge with the second media, and a third cartridge with a sorbent that includes quaternary amine functionality. In addition, the homogenously mixed sorbent media also outperforms a layered configuration of the first and second medias in a single cartridge. This result was unexpected. The microenvironment surrounding the sorbent beads yields the synergistic effects observed by having different sorbent beads adjacent to one another.

Further, the homogenously mixed media also outperforms a single media with dual functionality. This was a surprising result and demonstrates the synergistic effect of homogenously mixing the two types of media. In more detail, a homogenous mixture of a first media that includes hydroxyl functional groups and a second media that includes secondary and tertiary amine groups outperforms a single media that includes both hydroxyl, secondary amine, and tertiary amine groups. This was also an unexpected result. The result further illustrates the synergistic effect of homogenously mixing two types of sorbents, even compared to single sorbent with similar functional groups.

In addition, the homogenously mixed media is effective even under high-salinity conditions. One of the major challenges in the analysis of produced water samples from oil reservoirs is the extremely high salinity of the produced water. For connate water in reservoirs, the salinity can be as high as 240,000 ppm of total dissolved solids. The dissolved ions can include cations, for example Ca²⁺, Mg²⁺, Na⁺, K⁺, Ba⁺, and Sr⁺. The dissolved ions can include anions, for example, Cl⁻, Br⁻, SO₄²⁻, and CO₃²⁻. The dissolved ions can include other ions typically found in produced waters. In the produced water samples, even diluted with flooding fluids (i.e., fresher water or seawater), the salinity is usually higher than 100,000 ppm. The high salinity could obfuscate the measurements of most analytical techniques. Accordingly, in addition to preconcentration of tracer molecules, an ideal SPE process needs to remove these salts from the final eluate.

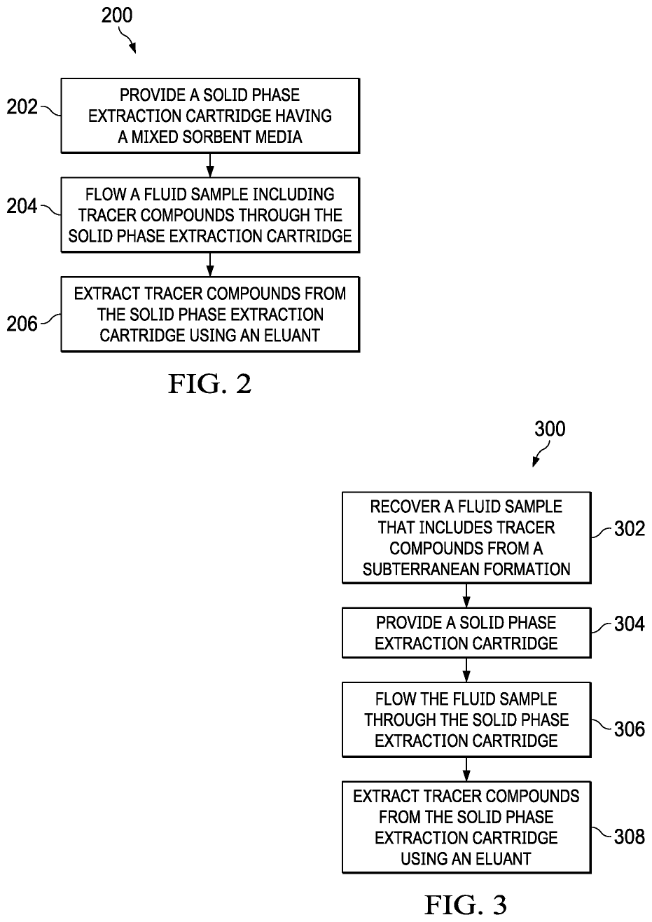

FIG. 2 shows an example flowchart of a method 200 of extracting tracer compounds from a fluid sample. At 202, a solid phase extraction cartridge having a mixed sorbent media is provided. The solid phase extraction cartridge includes an extraction column, a filter at the bottom of the extraction column, and a mixed sorbent media in the extraction column. The mixed sorbent media includes a homogenous mixture of a first sorbent and a second sorbent. The first sorbent includes a first polymer core, where the first polymer core includes a crosslinked polystyrene divinyl benzene copolymer functionalized with a hydroxylated polystyrene divinyl benzene copolymer. The second sorbent includes a second polymer core, where the second polymer core includes a crosslinked polystyrene divinyl benzene copolymer core functionalized with a copolymer that includes secondary and tertiary amino groups. At 204, the fluid sample is flowed through the solid phase extraction cartridge. At 206, the tracer compounds are extracted from the solid phase extraction cartridge using an eluent.

FIG. 3 shows an example flowchart of a method 300 of recovering tracers from a subterranean fluid. At 302, a fluid sample that includes tracer compounds is recovered from a subterranean formation. At 304, a solid phase extraction cartridge is provided. The SPE cartridge includes an extraction column, a filter at the bottom of the extraction column, and a solid phase media in the extraction column. The solid phase media includes a homogenous mixture of a first sorbent and a second sorbent. The first sorbent includes a first polymer core, where the first polymer core includes a crosslinked polystyrene divinyl benzene copolymer functionalized with a hydroxylated polystyrene divinyl benzene copolymer. The second sorbent includes a second polymer core, where the second polymer core includes a crosslinked polystyrene divinyl benzene copolymer core functionalized with a copolymer that includes secondary and tertiary amino groups. At 306, the fluid sample is flowed through the solid phase extraction cartridge. At 308, the tracer compounds are extracted from the solid phase extraction cartridge using an eluent.

### Example 1: Recovery of Tracers in Single Mode SPE Extraction

A deionized water sample containing 10 ppm each of dipicolinic acid (DPA), chelidamic acid (CDA), 4-chloropyridine dicarboxylic acid (Cl-DPA), and 1,5-napthalene disulfonate disodium (1,5-NDS) was prepared. This tracer sample was passed over a 6 mL SPE cartridge including either 200 mg of ENV+ SPE hydrophobic reverse-phase media or 500 mg of HR-XAW weak basic anion exchange SPE media, and the flow-through was discarded. Next, the tracers were extracted using methanol and 25 mM ammonia in methanol. The resulting eluates were pooled and analyzed using HPLC UV/Vis spectroscopy. The recovery of each of the tracers is shown in Table 1.

TABLE 1
Recovery of Tracers in Single-Mode SPE Cartridges

| | ENV+ | HR-XAW |
|---|---|---|
| | Recovery | Recovery |
| | Factor (%) | Factor (%) |
| 1,5-NDS | 1 | 100 |
| CDA | 43 | 65 |
| DPA | 86 | 81 |
| Cl-DPA | 59 | 100 |

### Example 2: Recovery of Traces in Sequential SPE Extraction

A tracer sample including 10 ppm of DPA, Cl-DPA, and 1,5-NDS in deionized water was prepared. Next, 0.5 mL of 6 N HCl was added to the tracer solution. 100 mL of the tracer solution was passed over a 6 mL, 200 mg ENV+ column. The flow through was collected. The tracers were eluted from the column with methanol and collected separately. A 6 mL, 500 mg HR-XAW column was preconditioned with 25 mM ammonium acetate in deionized water. The tracer solution recovered from the ENV+ column was passed over the HR-XAW column and the flow through was collected. The tracers were eluted from the HR-XAW column with methanol and 25 mM ammonia in methanol. A 6 mL, 500 mg HR-XA column was preconditioned with 1 M NaOH. The HR-XA column includes a strong anionic exchanger sorbent with quaternary ammonium functionality. The flow through from the HR-XAW column was passed over the HR-XA column. The tracers were eluted from the HR-XA column with methanol and methanolic acid. The final sample containing the eluents from all three columns were combined, dried, and redissolved in 1 mL of deionized water to preconcentrate the tracers. The final sample including the recovered tracers was then diluted 100× with deionized water and analyzed.

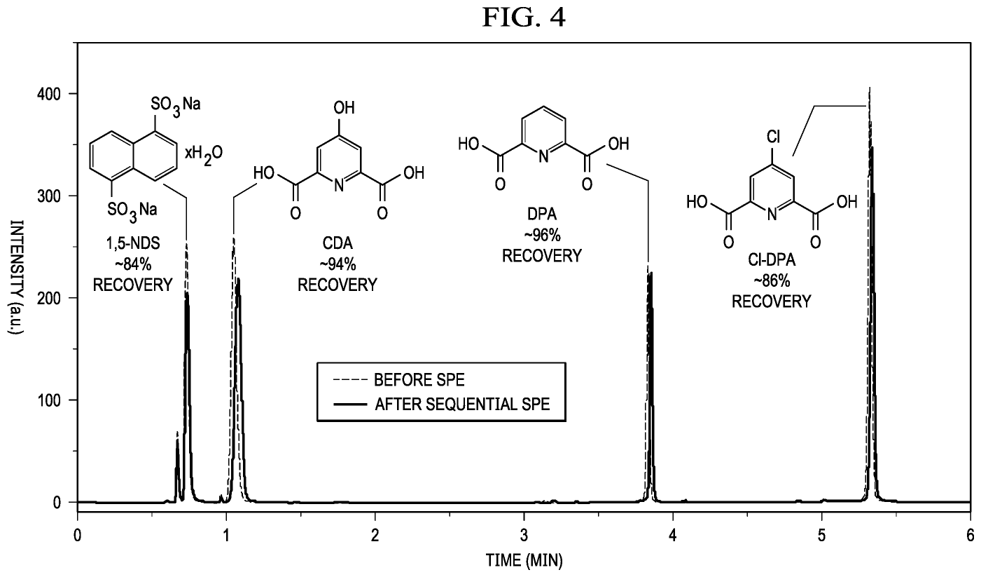

The recovered tracers were analyzed with HPLC, using a C18 column run on a gradient mode from 3% methanol in an acetate buffer to 30% methanol in an acetate buffer over 16 minutes. The column was then flushed with methanol for 5 minutes. Due to the increased viscosity of high methanol concentrations with the acetate buffer, the buffer pump was programmed to stop during the flushing part of the run to avoid over-pressurizing the device. The pump was programmed to resume during the post-run equilibration for 2 minutes, to allow flow to stabilize before the next run. The recovery percentages of the sequential extraction are shown in Table 2. FIG. 4 shows an example HPLC UV/Vis chromatogram at 214 nm of the water sample before and after sequential SPE extraction.

TABLE 2
Recovery of Tracers in Sequential SPE Cartridges

| | ENV+/HR-XAW/ |
|---|---|
| | HR-XA Sequential |
| | Recovery Factor (%) |
| 1,5-NDS | 84 |
| CDA | 94 |
| DPA | 96 |
| Cl-DPA | 86 |

### Example 3: Recovery of Tracers in Layered SPE Extraction

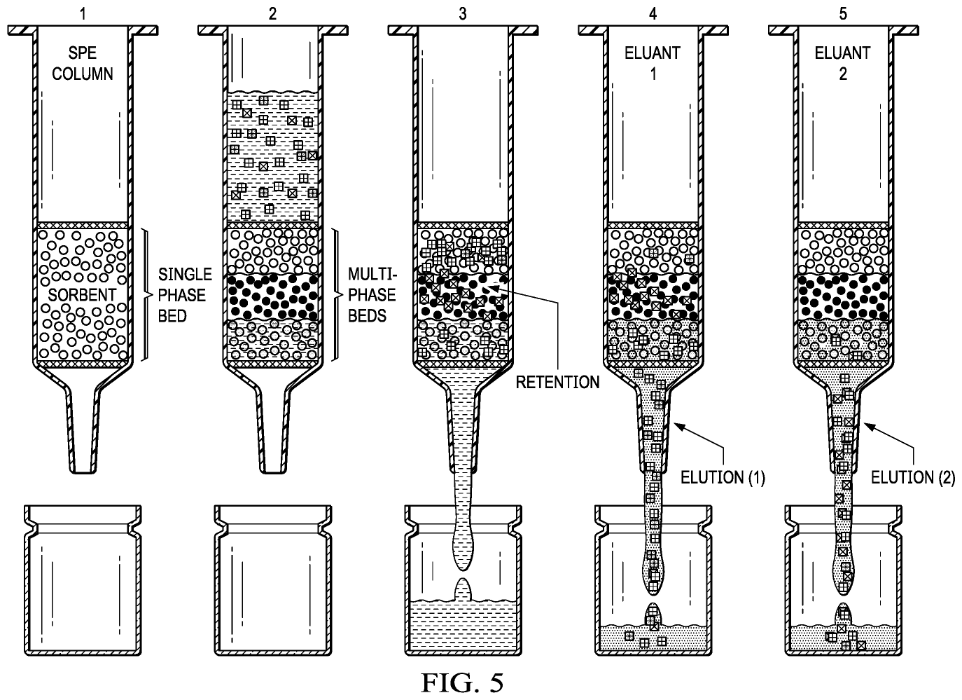

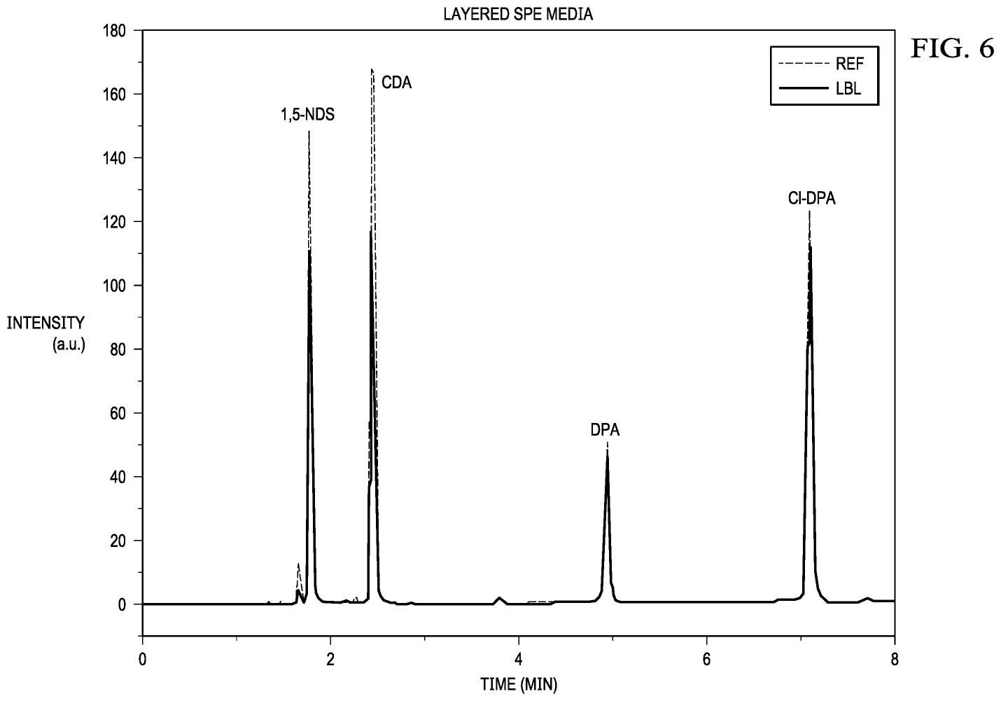

A layered SPE cartridge was prepared by placing a polyethylene frit (filter) with 10 μm porosity at the bottom of a cartridge, adding 200 mg of ENV+ sorbent, packing the sorbent with a second polyethylene frit (filter), placing 500 mg of HR-XAW on top of the second polyethylene frit, and compressing the HR-XAW sorbent. The tracer sample was passed over the layered SPE cartridge, and the eluent was discarded. FIG. 5 shows an example schematic of a layered SPE cartridge. Compared to a single-phase bed as shown in (1), the SPE cartridge as shown in (2) contains multiple layers of different types of SPE sorbents. The schematic in (2) shows three layers of sorbents, however, in this example, only two layers of sorbents were used. As shown in FIG. 5 (3), the tracers are retained in the sorbent layers, following by extraction with a first eluent in (4) and a second eluent in (5). In this example, a tracer sample including 10 ppm of DPA, Cl-DPA, CDA, and 1,5-NDS tracers in deionized water was prepared. Next, 0.5 mL of 6 N HCl was added to the tracer solution. The layered ENV+/HR-XAW SPE cartridge was preconditioned with 25 mM ammonium acetate in deionized water. Next, 100 mL of the tracer sample was passed over the ENV+/HR-XAW layered cartridge. The tracers were extracted from the layered ENV+/HR-XAW SPE using methanol and 25 mM ammonia in methanol. The resulting eluates were dried completely at 85° C. under N₂ flow for two and a half hours. The dried samples were then dissolved in 1 mL of deionized water and diluted 100× with deionized water. The diluted sample was analyzed using HPLC, using a C18 column run on a gradient mode from 3% methanol in an acetate buffer to 30% methanol in an acetate buffer over 16 minutes. The column was then flushed with methanol for 5 minutes. Due to the increased viscosity of high methanol concentrations with the acetate buffer, the buffer pump was programmed to stop during the flushing part of the run to avoid over-pressurizing the device. The pump was programmed to resume during the post-run equilibration for 2 minutes, to allow flow to stabilize before the next run. FIG. 6 shows an example HPLC UV/Vis absorption analysis of the recovery of the tracers at 214 nm, where the REF line represents an HPLC UV/Vis chromatogram of the tracer sample before extraction, and the LBL line represents eluted tracers after extraction the layer-by-layer ENV+/HR-XAW layered cartridge. The recovery factors of the layered SPE cartridge are shown in Table 3.

TABLE 3
Recovery of Tracers in Layered SPE Cartridge

| | ENV+/HR-XAW |
|---|---|
| | Layered Recovery |
| | Factor (%) |
| 1,5-NDS | 76 |
| CDA | 69 |
| DPA | 93 |
| Cl-DPA | 92 |

### Example 4: Recovery of Tracers in Homogenously Mixed-Mode SPE Extraction

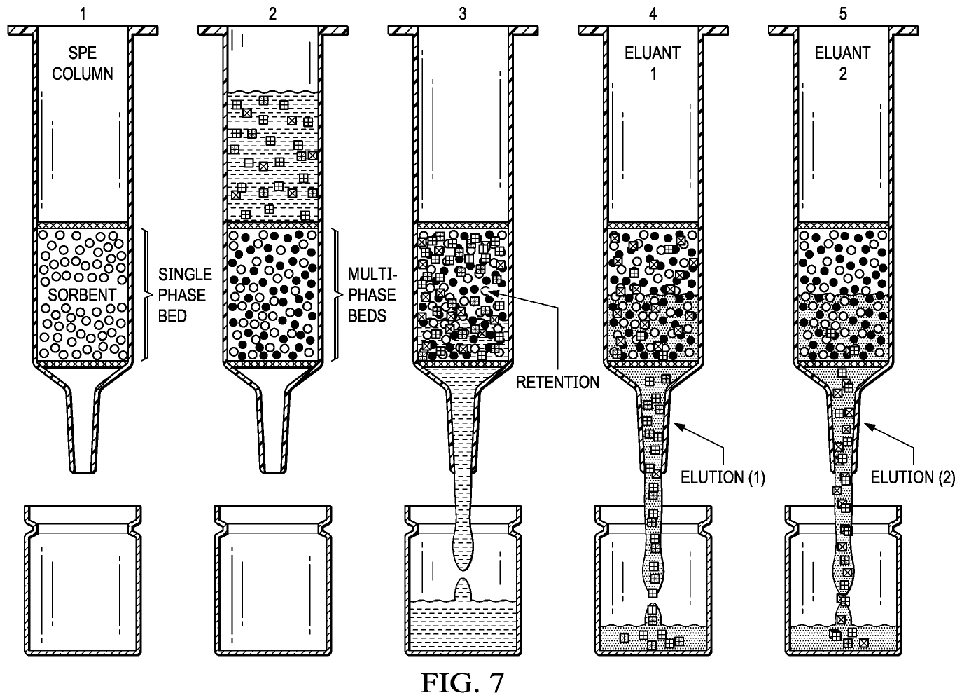

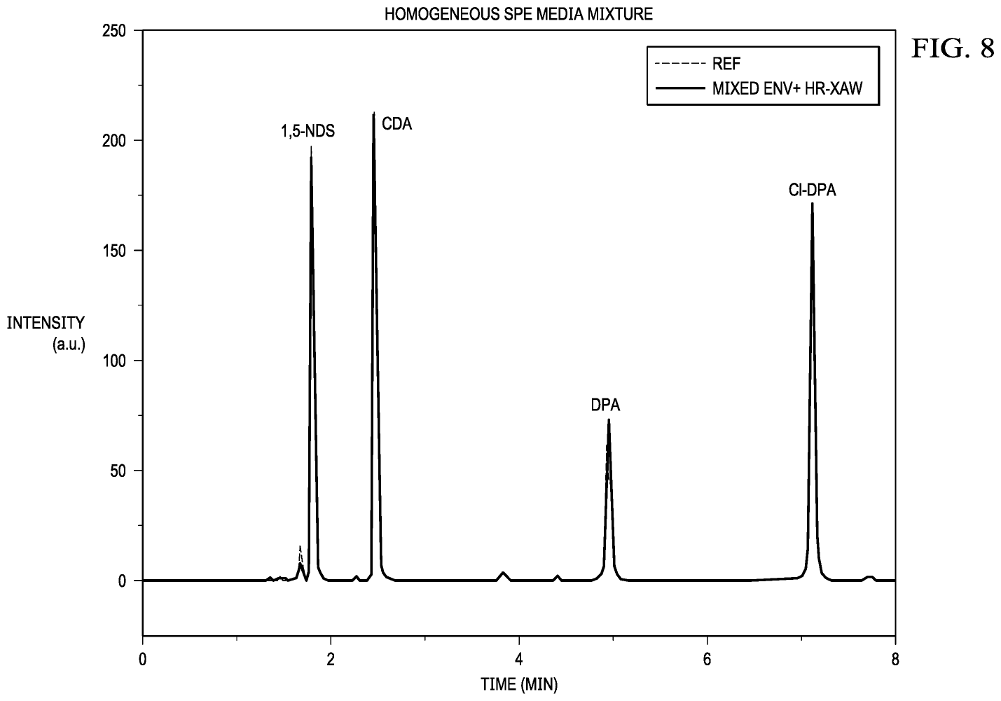

FIG. 7 shows an example schematic of an SPE extraction using a homogenously mixed solid phase bed. In contrast to the single-phase bed as shown in (1), the SPE cartridge used in this example contains a homogenously mixed bed, as shown in (2). The sample containing the tracers is loaded at step (2). At step (3), the tracers are retained in the homogenously mixed bed and the flow-through is discarded. At steps (4) and (5), the tracers are extracted with a first and second eluent. The resulting eluates are then pooled and analyzed.

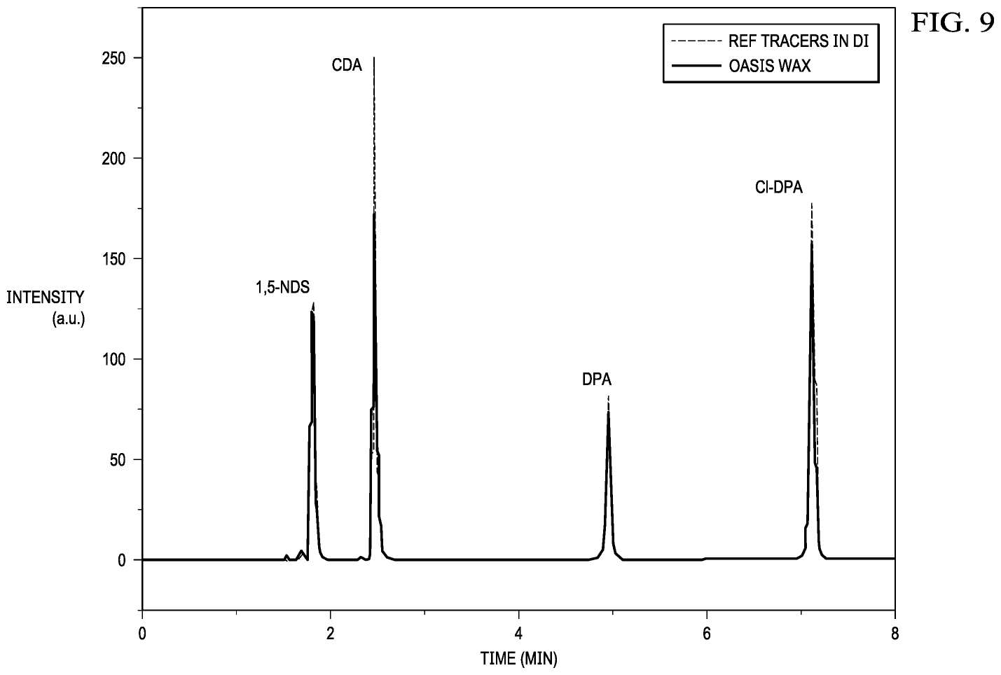

In this example, a deionized water sample containing the tracers was prepared as in Example 1. The sample was passed over a layered SPE cartridge containing a homogenous mixture of about 200 mg of ENV+ SPE media and about 500 mg of HR-XAW SPE media. The homogenously mixed cartridge was prepared by placing a polyethylene frit (filter) with 10 μm porosity at the bottom of the cartridge, adding the homogenously mixed sorbents, and covering the sorbents with a second polyethylene frit (filter). The sorbents were compressed by pressing on the second polyethylene frit. 100 mL of the tracer sample was passed over the homogenously mixed cartridge, and the flow-through was discarded. Next, the tracers were extracted using methanol and 25 mM ammonia in methanol. The extraction eluents were dried completely at 85° C. under N₂ flow for two and a half hours. The dried samples were then dissolved in 1 mL of deionized water and analyzed using HPLC, using a C18 column run on a gradient mode from 3% methanol in an acetate buffer to 30% methanol in an acetate buffer over 16 minutes. The column was then flushed with methanol for 5 minutes. Due to the increased viscosity of high methanol concentrations with the acetate buffer, the buffer pump was programmed to stop during the flushing part of the run to avoid over-pressurizing the device. The pump was programmed to resume during the post-run equilibration for 2 minutes, to allow flow to stabilize before the next run. FIG. 8 shows an example HPLC UV/Vis analysis at 214 nm of the combined eluents. Table 4 shows the recovery factors of the homogenously mixed SPE cartridge, where the REF line represents the tracer sample before elution and the MIXED ENV+HR-XAW line represents the eluted compounds after extraction with the homogenously mixed cartridge.

As shown in FIG. 8 and Table 4, all tracers of interest in the deionized water sample can be extracted with a near 100% recovery factor. In addition, these recovery factors are higher than the recovery factors of other configurations of SPE media. For example, chelidamic acid (CDA), was recovered from a homogenously mixed SPE cartridge at 98%. In contrast, CDA was only recovery at 69-93% for a sample that was passed through two different SPE cartridges sequentially or through a layered SPE cartridge.

Further, the recovery factor for each compound using the homogenously mixed SPE cartridges was greater than the recovery factors obtained using a mixed-mode Oasis WAX cartridge. The Oasis WAX cartridge contains an SPE media that is that displays both weak anion exchange and reverse phase properties on a single sorbent. FIG. 9 shows an example of an HPLC UV/Vis analysis at 214 nm of the recovery of tracer materials passed over an Oasis WAX cartridge, with 10 ppm DPA, Cl-DPA, CDA, and 1,5-NDS tracers in deionized water eluted with methanol and 25 mM ammonia in methanol. The recovery percentages of the Oasis WAX cartridge are shown in Table 4. As shown in Table 4, the homogenously mixed ENV+/HR-XAW cartridge outperformed the mixed mode Oasis WAX cartridge for three of the four tracers. For 1,5-NDS, the ENV+/HR-XAW and Oasis WAX cartridges performed similarly.

TABLE 4
Recovery of Tracers in Homogenously Mixed
ENV+/HR-XAW and Oasis WAX Cartridge

| | ENV+/HR-XAW | |
|---|---|---|
| | Homogenous | Oasis WAX |
| | Recovery Factor (%) | Recovery Factor (%) |
| 1,5-NDS | 97 | 100 |
| CDA | 98 | 68 |
| DPA | 100 | 91 |
| Cl-DPA | 100 | 89 |

### Example 5: Recovery of Tracers in Oil Field Produced Water

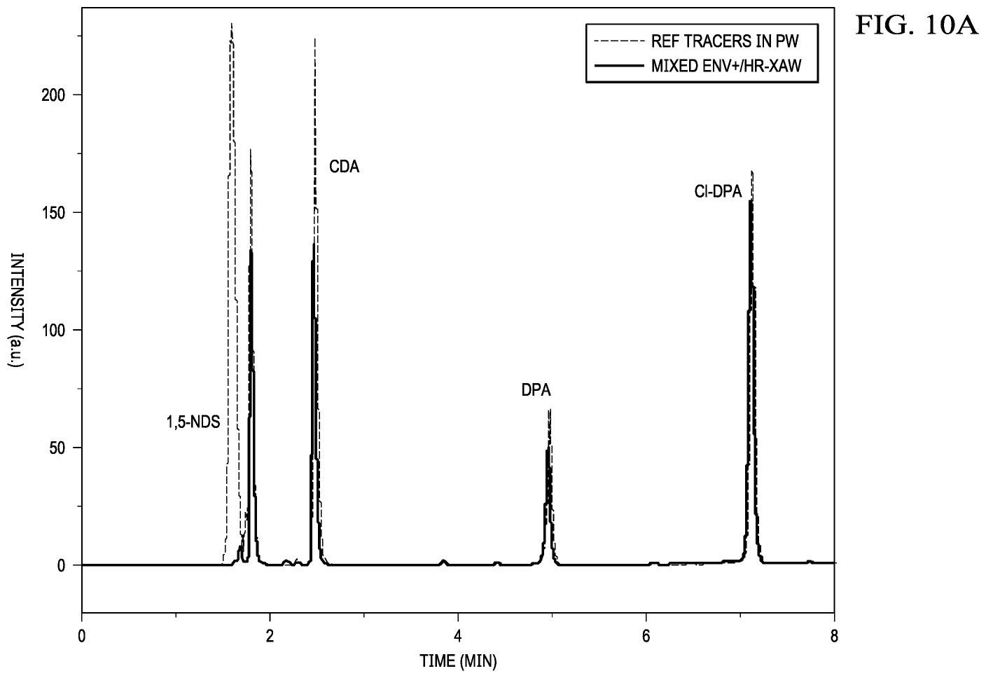

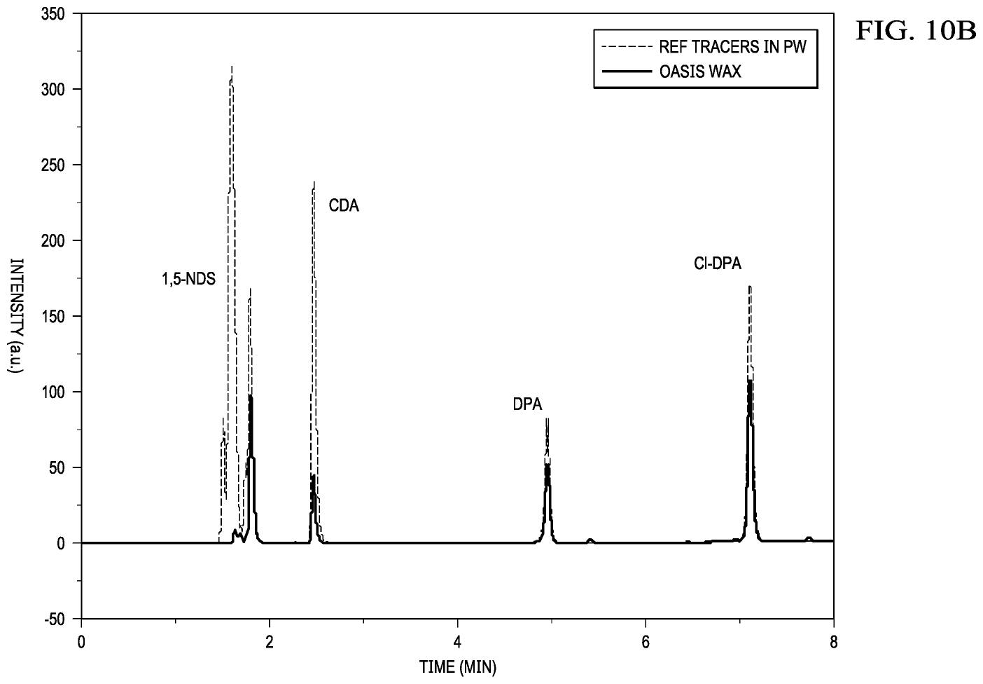

FIGS. 10A-10B shows an example of using the homogenously mixed mode ENV+/HR-XAW SPE cartridge compared to the Oasis WAX cartridge to recover tracers dissolved in oil field produced water. FIG. 10A shows an example HPLC UV/Vis analysis at 214 nm of recovered tracers in oil field produced water, recovered by a homogenously mixed ENV+/HR-XAW SPE cartridge, compared to a reference sample of tracers in produced water. The REF line represents a prepared sample of 10 ppm DPA, Cl-DPA, CDA, and 1,5-NDS in an oil field produced water, before extraction with an SPE cartridge. The MIXED ENV+/HR-XAW line represents the recovery of these tracers from the oil field water after extraction with the homogenously mixed mode cartridge and elution with methanol and 25 mM ammonia in methanol. FIG. 10B shows an example HPLC UV/Vis analysis at 214 nm of recovered tracers in oil field produced water, recovered by an Oasis WAX SPE cartridge, compared to a reference sample of tracers in produced water. The REF line represents a prepared sample of 10 ppm DPA, Cl-DPA, CDA, and 1,5-NDS in an oil field produced water, before extraction. The OASIS WAX line represents the recovery of these tracers from the oil field water after extraction with the Oasis WAX cartridge and elution with methanol and 25 mM ammonia in methanol. Table 5 shows the recovery factors for each of the tracer materials recovered by either the ENV+/HR-XAW homogenously mixed cartridge or the Oasis WAX cartridge.

TABLE 5
Percent Recovery of Tracers in Oil Field Produced Water

| | ENV+/HR-XAW | Oasis WAX |
|---|---|---|
| Tracer | Recovery Factor (%) | Recovery Factor (%) |
| 1,5-NDS | 68 | 60 |
| CDA | 58 | 19 |
| DPA | 72 | 62 |
| Cl-DPA | 92 | 64 |

As shown in Table 5, the homogenously mixed ENV+/HR-XAW outperforms the Oasis WAX cartridge. Compared to tracers in deionized water, the homogenously mixed ENV+/HR-XAW SPE cartridge recovers less of each tracer from the produced water. This decrease in recovery factors compared to recovery factors in deionized water may be attributed to the high salinity and abundance of dissolved organic compounds in the produced water, which in turn can affect the capacities of the extraction media. However, the homogenously mixed ENV+/HR-XAW SPE cartridge outperformed the Oasis WAX SPE cartridge.

### Example 6: Conductance of Solutions Before and After SPE Processes

The interaction of salt ions with the SPE cartridges (ENV+/HR-XAW and Oasis WAX) was studied. As discussed herein, the removal of salts from produced waters is beneficial for accurately analyzing the amount of a tracer compound in a produced water. The results of this analysis show that the homogenously mixed ENV+/HR-XAW SPE cartridge can efficiently remove a number of salts during an SPE process. Table 6 shows the conductance of solutions in micro Siemens (μS) before and after an SPE process.
The SPE process includes the following steps. The homogenously mixed ENV+/HR-WAX SPE cartridge and the Oasis WAX SPE cartridge were each first conditioned by 5 mL of methanol, followed by 5 mL of deionized (DI) water. 100 mL of test solution was used for the SPE process. The test solution was one of deionized water, synthetic seawater (seawater) or low-salinity brine (LS brine). The synthetic seawater includes about 66,000 ppm total dissolved solids. The low salinity brine includes about 136,000 ppm total dissolved solids. The conductivity of each of these samples was measured. The conductivities of solutions were measured with Brookhaven 90Plus/PALS zeta potential analyzer. Samples were placed in a cuvette with electrodes, and the sample conditions were remarked in Table 6. The prepared tracer solutions were passed over an ENV+, HR-XAW, or homogenously mixed ENV+/HR-XAW SPE cartridge and the flow through (A) was collected. The flow through (A) from each cartridge was analyzed for conductivity. The seawater and LS brine flow through (A) of the ENV+, HR-XAW, and homogenously mixed ENV+/HR-XAW cartridges each demonstrated high conductivity, indicating low retention of the salt ions on the cartridge. The cartridges were then washed with 5 mL of deionized water and the flow through (B) was collected. The flow through (B) demonstrated low conductivity, indicating low retention of salt ions on the cartridges, i.e., that the salt ions did not remain on the cartridge after the first flow through (A). Next, an eluate was collected with 5 mL of methanol. The eluted samples were dried completely at 85° C. under N₂ flow for two hours and half under N₂ gas flow, re-dissolved in 1 ml of DI water, and then diluted 100× to check recovery factors. The diluted sample was analyzed for conductance. The diluted sample of each cartridge demonstrated low conductivity, indicating low retention of salt ions on the SPE cartridges.

TABLE 6
Conductance of solutions (μS), before and after the SPE process

| | Conductance, μS | | | | |
|---|---|---|---|---|---|
| | | | | Mixed | |
| | | | | Mode | |
| | | | HR- | ENV+/ | |
| Solution | Blank | ENV+ | XAW | HR-XAW | Remark |
| DI water | 14 | | | | Samples before |
| Seawater | 61887 | | | | SPE Extraction |
| LS brine | 72681 | | | | |
| DI water | | 27 | 30 | 30 | Flow through (A) |
| Seawater | | 73041 | 77796 | 74279 | of samples after |
| LS brine | | 77494 | 88043 | 88251 | SPE Extraction |
| Seawater | | 127 | 102 | 81 | Flow through (B) |
| LS brine | | 540 | 78 | 72 | of DI water after |
| | | | | | SPE Extraction |
| Seawater | | 130 | 110 | 58 | Eluate of Methanol |
| LS brine | | 435 | 95 | 35 | after SPE Extrac- |
| | | | | | tion, dried and |
| | | | | | re-dissolved in |
| | | | | | DI water |

### Example 7: Recovery of Tracers in High Salinity Waters

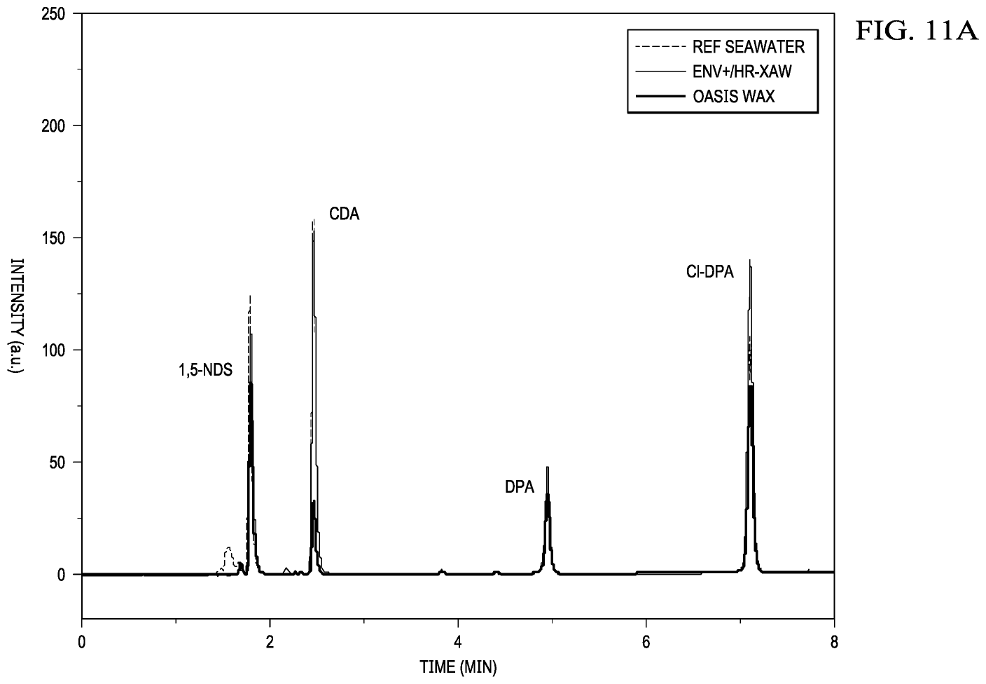

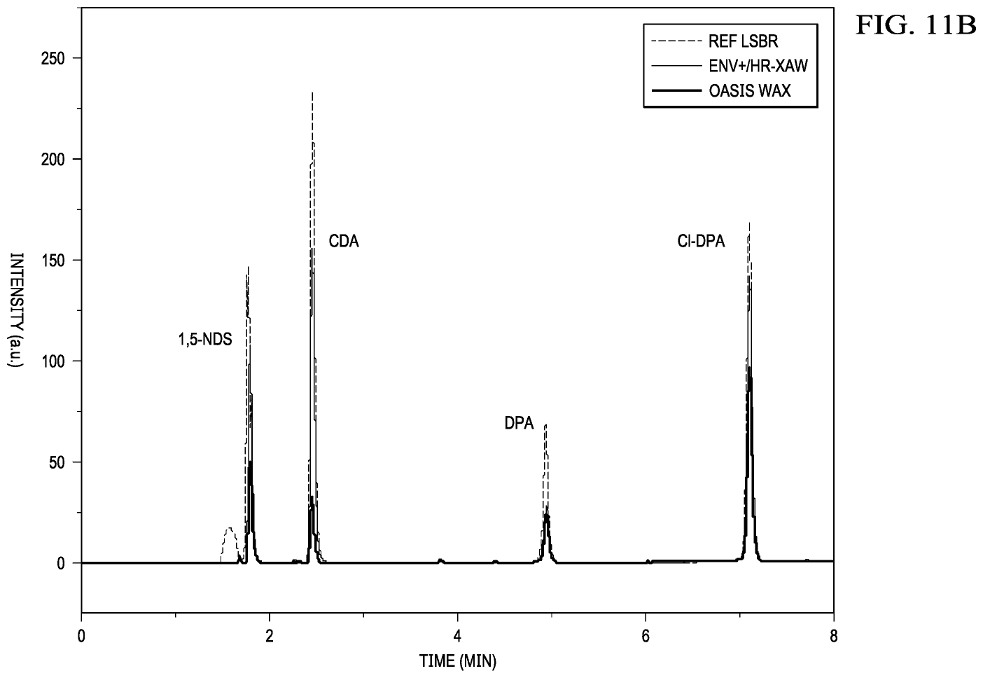

The influence of salinity on extraction efficiency was further investigated using synthetic seawater, with about 66,000 ppm total dissolved solids, and low salinity brine, with about 136,000 ppm total dissolved solids. As shown in FIGS. 11A-11B, the recovery factors of tracers in seawater were mostly consistent with what was observed for extraction efficiencies in deionized water, with minimal reductions in efficacies. However, as salinity increases, such as in the low salinity brine, a decrease in extraction efficiencies was observed. The decrease in efficiency may be the result of the high salt concentration interfering with the extraction of tracers. Even though this salt interference is observed, all of the results using a homogenously mixed mode ENV+/HR-XAW SPE cartridge showed superior extraction efficiencies compared to the Oasis WAX cartridge.

FIG. 11A shows an example HPLC UV/Vis analysis at 214 nm of seawater with tracers over a homogenously mixed ENV+/HR-XAW cartridge and an Oasis WAX cartridge. A solution containing 10 ppm of each of the tracers DPA, Cl-DPA, CDA, 1,5-NDS in 100 mL synthetic seawater solution was passed through the ENV+/HR-XAW Oasis WAX cartridge. The tracers were eluted by 5 ml methanol and 5 ml 2 M ammonia in methanol then dried completely at 85° C. under N₂ flow for two and a half hours. The dried samples were prepared by dissolving in 1 mL of DI water then diluted 100× in DI water to yield a diluted sample. The HPLC UV/Vis chromatogram at 214 nm of the 10 ppm tracer solution in seawater without the SPE extraction (REF SEAWATER) and with the SPE extraction process (diluted sample, ENV+/HR-XAW or OASIS WAX) is shown in FIG. 11A. FIG. 11B shows an HPLC UV/Vis chromatogram at 214 nm of the 10 ppm tracer solution in low salinity brine without SPE extraction (REF LSBR) and with the SPE extraction process (diluted sample, ENV+/HR-XAW or OASIS WAX). The recovery factors of each SPE extraction were calculated by comparing the original, unprocessed solution with the final diluted sample.

As shown in FIGS. 11A and 11B, and in Table 7, below, the homogenously mixed ENV+/HR-XAW SPE cartridge outperformed the Oasis WAX SPE cartridge.

TABLE 7
Recovery Factors of Tracers in Synthetic
Seawater and Low Salinity Brine

| | Synthetic Seawater | | Low Salinity Brine | |
|---|---|---|---|---|
| | ENV+/ | | ENV+/ | |
| | HR-XAW | Oasis WAX | HR-XAW | Oasis WAX |
| Tracer | Recovery | Recovery | Recovery | Recovery |
| Material | (%) | (%) | (%) | (%) |
| 1,5-NDS | 87 | 73 | 59 | 29 |
| CDA | 97 | 20 | 68 | 23 |
| DPA | 100 | 74 | 40 | 35 |
| Cl-DPA | 100 | 80 | 88 | 60 |

The term “about” as used in this disclosure can allow for a degree of variability in a value or range, for example, within 10%, within 5%, or within 1% of a stated value or of a stated limit of a range.

The term “substantially” as used in this disclosure refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “solvent” as used in this disclosure refers to a liquid that can dissolve a solid, another liquid, or a gas to form a solution. Non-limiting examples of solvents are silicones, organic compounds, water, alcohols, ionic liquids, and supercritical fluids.

The term “room temperature” as used in this disclosure refers to a temperature of about 15 degrees Celsius (° C.) to about 28° C.

As used in this disclosure, the term “subterranean material” or “subterranean zone” refers to any material under the surface of the earth, including under the surface of the bottom of the ocean. For example, a subterranean zone or material can be any section of a wellbore and any section of a subterranean petroleum- or water-producing formation or region in fluid contact with the wellbore. Placing a material in a subterranean zone can include contacting the material with any section of a wellbore or with any subterranean region in fluid contact the material. Subterranean materials can include any materials placed into the wellbore such as cement, drill shafts, liners, tubing, casing, or screens; placing a material in a subterranean zone can include contacting with such subterranean materials. In some examples, a subterranean zone or material can be any downhole region that can produce liquid or gaseous petroleum materials, water, or any downhole section in fluid contact with liquid or gaseous petroleum materials, or water. For example, a subterranean zone or material can be at least one of an area desired to be fractured, a fracture or an area surrounding a fracture, and a flow pathway or an area surrounding a flow pathway, in which a fracture or a flow pathway can be optionally fluidly connected to a subterranean petroleum- or water-producing region, directly or through one or more fractures or flow pathways.

As used in this disclosure, “treatment of a subterranean zone” can include any activity directed to extraction of water or petroleum materials from a subterranean petroleum- or water-producing formation or region, for example, including drilling, stimulation, hydraulic fracturing, clean-up, acidizing, completion, cementing, remedial treatment, abandonment, aquifer remediation, identifying oil rich regions via imaging techniques, and the like.

As used in this disclosure, “weight percent” (wt %) can be considered a mass fraction or a mass ratio of a substance to the total mixture or composition. Weight percent can be a weight-to-weight ratio or mass-to-mass ratio, unless indicated otherwise.

A number of implementations of the disclosure have been described. Nevertheless, it will be understood that various modifications may be made without departing from the spirit and scope of the disclosure.

What is claimed is:

1. A media for solid phase extraction, comprising:
a homogenous mixture of a first sorbent and a second sorbent, wherein
the first sorbent comprises a first polymer core, wherein the first polymer core comprises a crosslinked polystyrene divinyl benzene copolymer core functionalized with a hydroxylated polystyrene divinyl benzene copolymer, and
the second sorbent comprises a second polymer core, wherein the second polymer core comprises a crosslinked polystyrene divinyl benzene copolymer core functionalized with a copolymer that contains secondary and tertiary amino groups.

2. The media of claim 1, wherein the homogenous mixture of the first sorbent and the second sorbent comprises an equal amount of the first sorbent and the second sorbent.

3. The media of claim 1, wherein the homogenous mixture of the first sorbent and the second sorbent comprises a 2:5 ratio by weight of the first sorbent to the second sorbent.

4. A solid phase extraction cartridge, comprising:
an extraction column;
a filter at a bottom of the extraction column; and
a solid phase media in the extraction column, wherein the solid phase media comprises a homogenous mixture of a first sorbent and a second sorbent, wherein
the first sorbent comprises a first polymer core, wherein the first polymer core comprises a crosslinked polystyrene divinyl benzene copolymer core functionalized with a hydroxylated polystyrene divinyl benzene copolymer, and
the second sorbent comprises a second polymer core, wherein the second polymer core comprises a crosslinked polystyrene divinyl benzene copolymer core functionalized with a copolymer that contains secondary and tertiary amino groups.

5. The solid phase extraction cartridge of claim 4, wherein the homogenous mixture of the first sorbent and the second sorbent comprises an equal amount of the first sorbent and the second sorbent.

6. The solid phase extraction cartridge of claim 4, wherein the homogenous mixture of the first sorbent and the second sorbent comprises a 2:5 ratio by weight of the first sorbent to the second sorbent.

7. A method of extracting tracer compounds from a fluid sample, comprising:
providing a solid phase extraction cartridge, wherein the solid phase extraction cartridge comprises
an extraction column,
a filter at a bottom of the extraction column, and
a solid phase media in the extraction column, wherein the solid phase media comprises a homogenous mixture of a first sorbent and a second sorbent, wherein
the first sorbent comprises a first polymer core, wherein the first polymer core comprises a crosslinked polystyrene divinyl benzene copolymer core functionalized with a hydroxylated polystyrene divinyl benzene copolymer, and
the second sorbent comprises a second polymer core, wherein the second polymer core comprises a polystyrene divinyl benzene copolymer core functionalized with a copolymer that contains secondary and tertiary amino groups;
flowing the fluid sample through the solid phase extraction cartridge; and
extracting tracer compounds from the solid phase extraction cartridge using an eluent.

8. The method of claim 7, wherein extracting the tracer compounds from the solid phase extraction cartridge using an eluent comprises extracting the tracer compounds from the solid phase extraction cartridge using methanol.

9. The method of claim 7, further comprising extracting the tracer compounds from the solid phase extraction cartridge using a second eluent.

10. The method of claim 9, wherein extracting the tracer compounds from the solid phase extraction cartridge using a second eluent comprises extracting the tracer compounds from the solid phase extraction cartridge using a solution of ammonia in methanol.

11. The method of claim 7, further comprising conditioning the solid phase extraction cartridge with methanol before flowing the fluid sample through the solid phase extraction cartridge.

12. The method of claim 11, further comprising conditioning the solid phase extraction cartridge with deionized water before flowing a fluid sample through the solid phase extraction cartridge.

13. The method of claim 7, further comprising washing the solid phase extraction cartridge between flowing the fluid sample through the solid phase extraction cartridge and extracting the tracer compounds from the solid phase extraction cartridge using an eluent, wherein washing the solid phase extraction cartridge comprises flowing deionized water through the cartridge.

14. The method of claim 7, further comprising analyzing the extracted tracer compounds using HPLC, UV/Vis spectroscopy, or both.

15. A method of recovering tracers from a subterranean fluid, comprising:
recovering a fluid sample from a subterranean formation, wherein the fluid sample comprises tracer compounds;
providing a solid phase extraction cartridge, wherein the solid phase extraction cartridge comprises
an extraction column,
a filter at a bottom of the extraction column, and
a solid phase media in the extraction column, wherein the solid phase media comprises a homogenous mixture of a first sorbent and a second sorbent, wherein
the first sorbent comprises a first polymer core, wherein the first polymer core comprises a crosslinked polystyrene divinyl benzene copolymer core functionalized with a hydroxylated polystyrene divinyl benzene copolymer, and
the second sorbent comprises a second polymer core, wherein the second polymer core comprises a crosslinked polystyrene divinyl benzene copolymer core functionalized with a copolymer that contains secondary and tertiary amino groups;
flowing the fluid sample through the solid phase extraction cartridge; and
extracting the tracer compounds from the solid phase extraction cartridge using an eluent.
